(19) United States
(12) Patent Application Publication
(10) Pub. No.: US 2024/0350823 A1
(43) Pub. Date: Oct. 24, 2024

(54) Flexible light for skin therapy with adjustable attachment mechanism

(71) Applicant: BEDROCK BIOSCIENCE LLC, CLEARWATER, FL (US)

(72) Inventors: Vivienne Reign, Clearwater, FL (US); Philip Straw, Clearwater, FL (US); Jesse Harper, Wesley Chapel, FL (US)

(21) Appl. No.: 18/305,615

(22) Filed: Apr. 24, 2023

(51) Int. Cl. A61N 5/06

(52) U.S. Cl. CPC A61N 5/0616; A61N 2005/0632; A61N 2005/0645; A61N 2005/0652

(57) ABSTRACT

The flexible light for skin therapy with adjustable attachment mechanism includes a circuit board to which the multiplicity of LEDs is attached to form one or more LED arrays. The use of a flexible circuit board allows the electronic components to conform to the shape of the user's body, but also able to be shipped and stored in a flat, space-saving configuration. The silica gel encapsulates the LEDs, and is transparent to the frequency of light emitted by the LEDs. The body of the device includes perforated edges. The perforated edges are created from a multiplicity of holes arranged in line to create a perforated strip. Repositionable camlocks, or locks that hold straps in position, interface with the holes. Each repositionable camlock preferably includes mushroom-shaped pins, the head of each mushroom-shaped pin passing through a hole in the perforated strip.

### FIELD

This invention relates to the field of light therapy and more particularly to a flexible device for light therapy.

### BACKGROUND

Skin therapy using light is a growing field of medicine. Light therapy shows promise in treating a variety of skin conditions. For example, light therapy can treat conditions such as acne, psoriasis, and eczema.

One of the most common types of light therapy is LED light therapy. This therapy uses LED lights of different colors to target specific skin conditions. Blue light is commonly used to treat acne, while red light is used to reduce inflammation and promote healing.

LED light therapy is generally considered safe, with few side effects.

But devices that provide light therapy have historically been difficult to affix to the body while allowing the user to continue with other tasks.

What is needed is a device it is flexible for use on the body, with an adaptable strapping mechanism to allow the user to tailor the device to their particular needs.

### SUMMARY

The flexible light for skin therapy with adjustable attachment mechanism includes a circuit board to which the multiplicity of LEDs is attached to form one or more LED arrays.

The use of a flexible circuit board allows the electronic components to conform to the shape of the user's body, but also able to be shipped and stored in a flat, space-saving configuration.

The flexible circuit board is encapsulated within a flexible silica gel material. The material encapsulates the components and excludes moisture. The silica gel is transparent to the frequency of light emitted by the LEDs.

The preferred frequencies of light transmission are wavelengths of 660 nm and 880 nm.

The body of the device includes perforated edges. The perforated edges are created from a multiplicity of holes arranged in line to create a perforated strip. Repositionable camlocks, strap locks, or other means of holding straps in position, interface with the holes. This flexibility of camlock locations allows for a variety of associated strap locations and thus versatile application to the body.

Each repositionable camlock preferably includes mushroom-shaped pins, the head of each mushroom-shaped pin passing through a hole in the perforated strip

The straps optionally include preferred strap locations for various body parts. Such preferred strap locations are optionally marked along the perforated strip, along the user to readily locate suggested camlock placement.

The device is optionally used in conjunction with a disposable clear cover. The disposable clear cover is held in place using the camlocks, the mushroom-shaped heads of the camlocks passing through both the perforated strip and disposable clear cover, holding the two pieces in place with respect to each other.

The disposable clear cover allows the device to be readily sanitized between uses without requiring the use of caustic chemicals.

A timer, preferably separate from the device, is optionally available. The timer controls power to one or more devices.

This allows a user to wear multiple devices on various body parts, controlled by a single timer. The result is a reduction in the total time required for therapy.

An optional replaceable and rechargeable battery allows the device to be used without requiring a constant power source, such as receptacle.

An optional storage bag that includes internal sterilizing UV LEDs thoroughly sterilizes the device between uses.

An additional optional element is a reflective sleeve or cover, placed around the device after the device has been placed on the body. The cover is internally reflective, reducing the loss of light to the surrounding environment and therefore increasing the effectiveness of the therapy.

In addition to the above LED light emitters, the device optionally includes PEMF, or pulsed electromagnetic field emitters for pulsed electromagnetic field therapy. PEMF therapy uses electromagnetic fields to heal internal conditions. Such conditions include pain, inflammation, and poor circulation.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric of the flexible light for skin therapy with adjustable attachment mechanism.

FIG. 2 illustrates a second isometric, with the clear cover shown, of the flexible light for skin therapy with adjustable attachment mechanism.

FIG. 3 illustrates a detailed view of the top side of the strap lock of the flexible light for skin therapy with adjustable attachment mechanism.

4987.0 3

FIG. 4 illustrates a detailed view of the bottom side of the strap lock of the flexible light for skin therapy with adjustable attachment mechanism.

FIG. 5 illustrates an exploded view of the strap lock of the flexible light for skin therapy with adjustable attachment mechanism.

FIG. 6 illustrates a rear view of the flexible light for skin therapy with adjustable attachment mechanism.

FIG. 7 illustrates an isometric of the upper section of the flexible light for skin therapy with adjustable attachment mechanism.

FIG. 8A illustrates a first view of the timer of the flexible light for skin therapy with adjustable attachment mechanism.

FIG. 8B illustrates a second view of the timer of the flexible light for skin therapy with adjustable attachment mechanism.

FIG. 9A illustrates a first view of the storage case of the flexible light for skin therapy with adjustable attachment mechanism.

FIG. 9B illustrates a second view of the storage case of the flexible light for skin therapy with adjustable attachment mechanism.

FIG. 9C illustrates a third view of the storage case of the flexible light for skin therapy with adjustable attachment mechanism.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

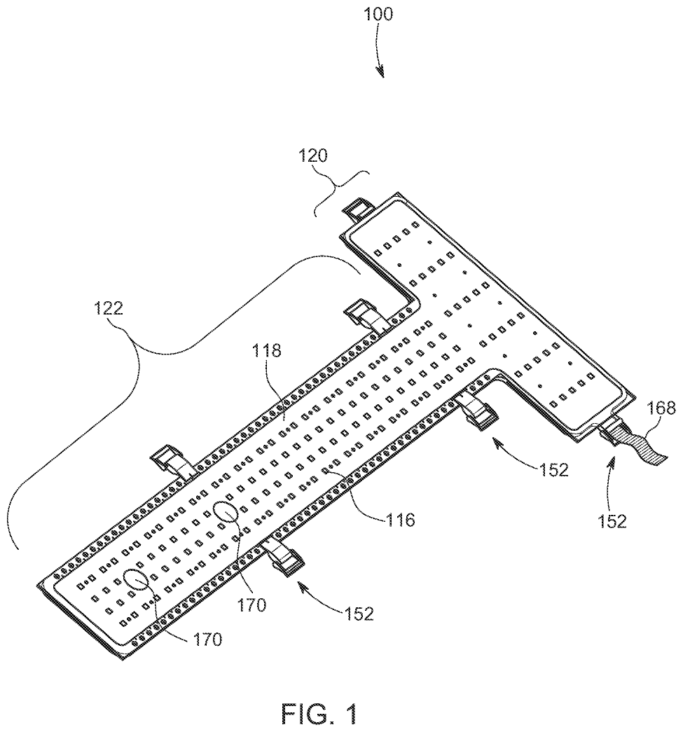

Referring to FIG. 1, a first isometric of the flexible light for skin therapy with adjustable attachment mechanism is shown.

The flexible light for skin therapy with adjustable attachment mechanism 100 includes a first module 120, and a second module 122, that in combination preferably form a T-shape. This shape is versatile, allowing for application of the device to many different parts of the body.

Each module 120/122 includes a multiplicity of light-emitting elements 116 that as a group form a light-emitting array 118.

Strap locks 152 interact with straps 168 to allow the flexible light for skin therapy with adjustable attachment mechanism 100 to be attached to the body.

Also shown are the one or more PEMF emitters 170, or pulsed electromagnetic field emitters for pulsed electromagnetic field therapy. PEMF therapy uses electromagnetic fields to heal internal conditions. Such conditions include pain, inflammation, and poor circulation.

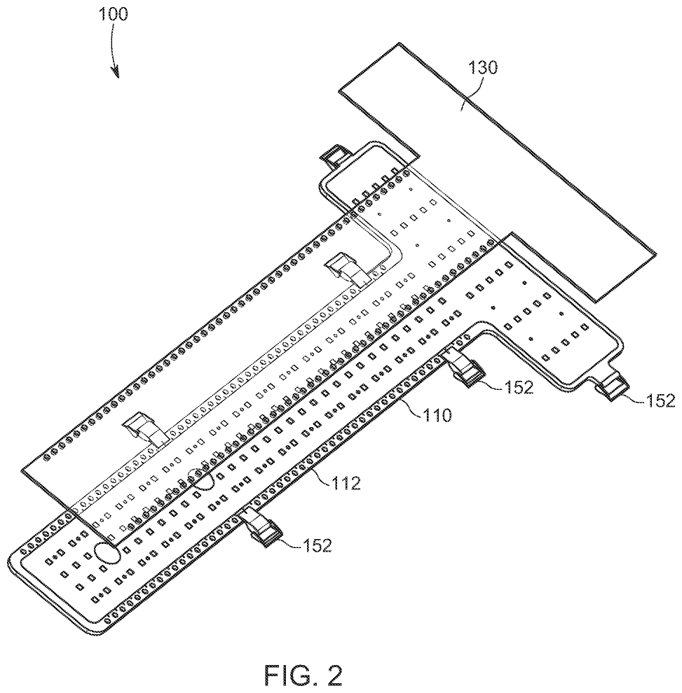

Referring to FIG. 2, a second isometric, with the clear cover shown, of the flexible light for skin therapy with adjustable attachment mechanism is shown.

An optional protective membrane 130, or disposable cover, is used with the device. The protective membrane 130 can be removed from the body 110 and replaced between patients, eliminating risk of cross contamination.

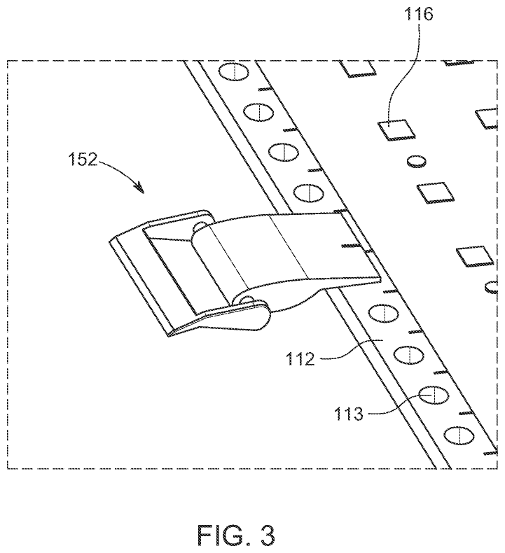

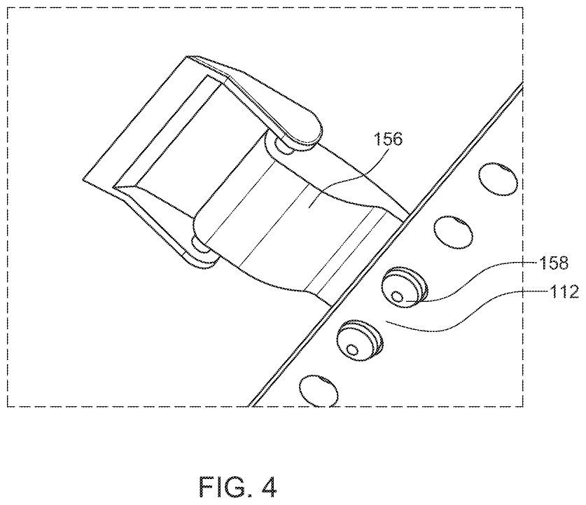

Referring to FIGS. 3 and 4, a detailed view of the top side and bottom side of the strap lock of the flexible light for skin therapy with adjustable attachment mechanism are shown.

Shown is one strap lock 152 snapped into the perforated edge 112 with holes 113. Strap block 152 snaps into the perforated edge 112 via a repositionable tab 156 that includes one or more mushroom-shaped heads 158. The use of two or more mushroom-shaped heads 158 is preferred to avoid rotation of the strap lock 152 after placement.

The light-emitting elements 116 are visible adjacent to the perforated edge 112.

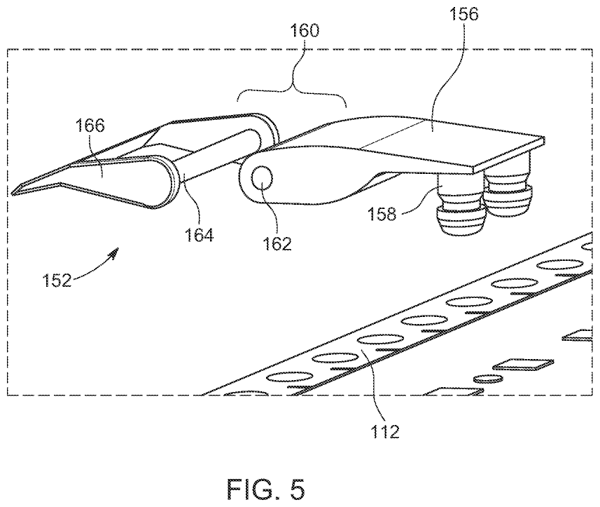

Referring to FIG. 5, an exploded view of the strap lock of the flexible light for skin therapy with adjustable attachment mechanism is shown.

The strap lock 152 shown includes a repositionable tab 156 with one or more mushroom-shaped heads 158.

A hinge 160 is formed from a hole 162 and pin 164, ending in a locking lever 166.

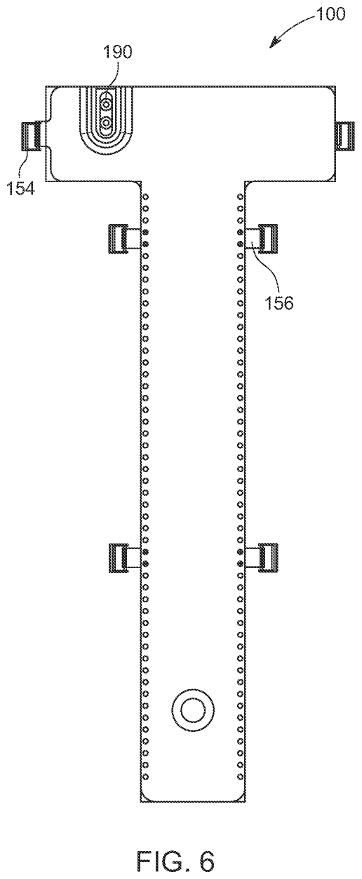

Referring to FIG. 6, a rear view of the flexible light for skin therapy with adjustable attachment mechanism is shown.

The preferred embodiment includes both fixed tabs 154 and repositionable tabs 156.

Also shown on the rear of the flexible light for skin therapy with adjustable attachment mechanism 100 is a power receptacle 190.

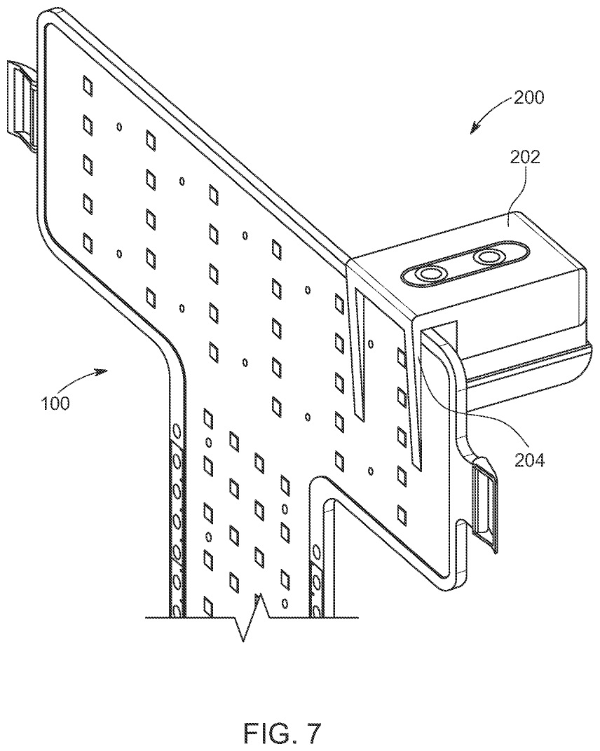

Referring to FIG. 7, an isometric of the upper section of the flexible light for skin therapy with adjustable attachment mechanism is shown.

In this embodiment, the power source 200 is shown as a battery pack 202. The battery pack 202 uses battery pack hooks 204 to hang over the flexible light for skin therapy with adjustable attachment mechanism 100.

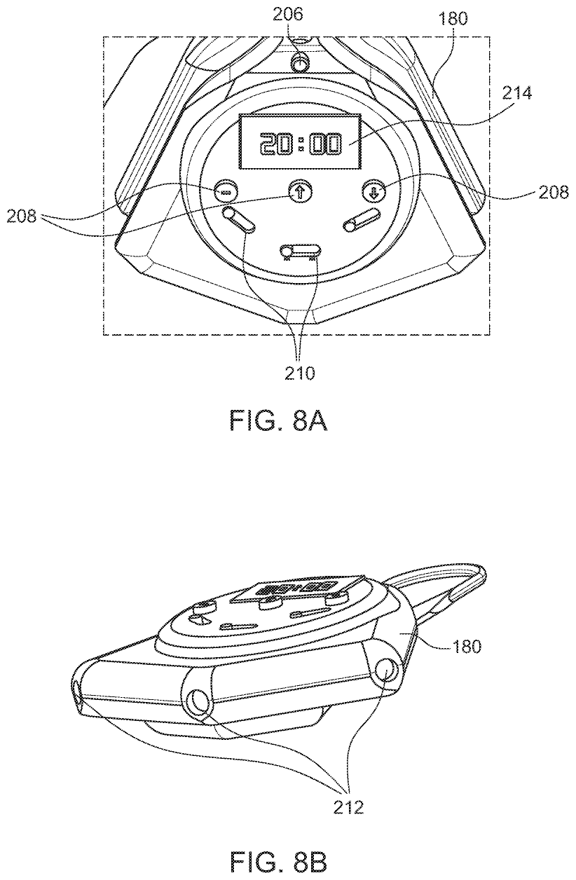

Referring to FIGS. 8A and 8B, a first view and a second view of the timer of the flexible light for skin therapy with adjustable attachment mechanism are shown.

The controller 180 is shown with power indicator 206, input buttons 208, power switches for individual ports 210, display 214, and power ports 212. Each power port 212 can be connected to a single flexible light for skin therapy with adjustable attachment mechanism 100 (see FIG. 1).

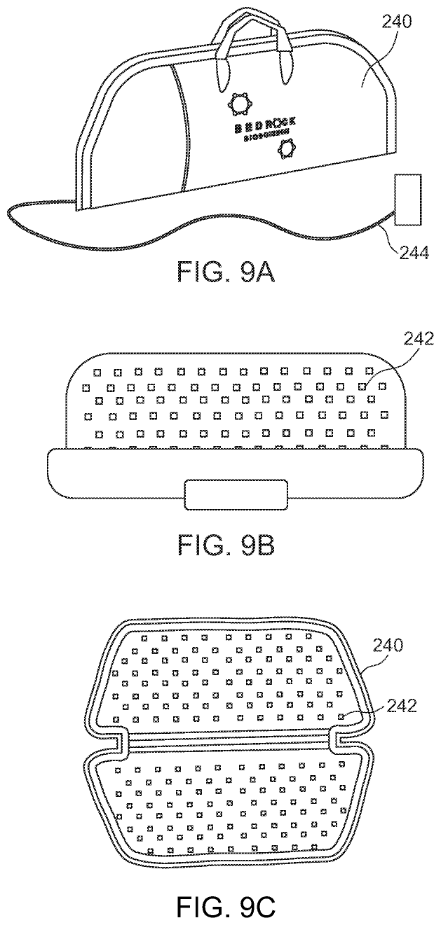

Referring to FIGS. 9A, 9B, and 9C, a first, second, and third view of the storage case of the flexible light for skin therapy with adjustable attachment mechanism are shown.

The cleaning case 240 includes interior ultraviolet disinfecting LEDs 242 for sanitization of a flexible light for skin therapy with adjustable attachment mechanism 100 (see FIG. 1). Power is provided by power adapter 244.

Equivalent elements can be substituted for the ones set forth above such that they perform in substantially the same manner in substantially the same way for achieving substantially the same result.

What is claimed is:

1. A flexible light therapy device comprising:
a body with one or more perforated edges;
the one and more perforated edges acting as strap attachment points;
an LED array for emitting therapeutic light;
the LED array integrated into the body;
a repositionable strap lock formed from a repositionable tab with one or more mushroom-shaped heads;
the one or more mushroom-shaped heads removably locking into holes of the one or more perforated edges, fixing the repositionable strap lock in a temporary location;
the repositionable strap lock connected to a repositionable strap;
whereby the repositionable strap lock can be moved to best suit an intended use.

2. The flexible light therapy device of claim 1, further comprising:
a disposable cover;
the disposable cover placed over the body;
the disposable cover including one or more perforated cover edges that line up with the one or more perforated edges of the body;
the disposable cover held in position with respect to the body by the repositionable strap lock;
whereby when the repositionable strap lock is removed, the disposable cover is released from the body for disposal.

3. The flexible light therapy device of claim 1, wherein:
the one or more perforated edges is a first perforated edge and a second perforated edge;
the first perforated edge is parallel to the second perforated edge;
the repositionable strap crossing from the first perforated edge to the second perforated edge to affix the device in position.

4. The flexible light therapy device of claim 3, further comprising:
a disposable cover;
the disposable cover placed over the body;
the disposable cover including one or more perforated cover edges that line up with the one or more perforated edges of the body;
the disposable cover held in position with respect to the body by the repositionable strap lock;
whereby when the repositionable strap lock is removed, the disposable cover is released from the body for disposal.

5. The flexible light therapy device of claim 1, further comprising:
a removable timer;
the removable timer electrically connected to the LED array;
the removable timer controlling whether the LED array is emitting light;
whereby the removable timer simplifies use.

6. A device for use during light therapy of skin, the device comprising
an LED array embedded within a flexible body
a first perforated edge and a second perforated edge along the flexible body;
a first repositionable strap lock and a second repositionable strap lock;
the first repositionable strap lock affixed to the first perforated edge;
the second repositionable strap lock affixed to the second perforated edge;
the first repositionable strap lock connected to the second repositionable strap lock by a strap;
whereby changing r location of the first repositionable strap lock and the second repositionable strap lock changes a position of the strap, thereby allowing the device to be used in various orientations.

7. The device for use during light therapy of skin of claim 6, further comprising:
a disposable cover;
the disposable cover placed over the flexible body;
the disposable cover including one or more perforated cover edges that line up with the first perforated edge and the second perforated edge of the flexible body;
the disposable cover held in position with respect to the flexible body by the first repositionable strap lock and the second repositionable strap lock;
whereby when the first repositionable strap lock and the second repositionable strap lock are removed, the disposable cover is released from the flexible body for disposal.

8. The device for use during light therapy of skin of claim 6, wherein:
the first perforated edge is parallel to the second perforated edge;
the strap crossing from the first perforated edge to the second perforated edge to affix the device in position.

9. The device for use during light therapy of skin of claim 8, further comprising:
a disposable cover;
the disposable cover placed over the flexible body;
the disposable cover including one or more perforated cover edges that line up with the first perforated edge and the second perforated edge;
the disposable cover held in position with respect to the flexible body by the first repositionable strap lock and the second repositionable strap lock;
whereby when the first repositionable strap lock and the second repositionable strap lock are removed, the disposable cover is released from the flexible body for disposal.

10. The device for use during light therapy of skin of claim 6, further comprising:
a removable timer;
the removable timer electrically connected to the LED array;
the removable timer controlling whether the LED array is emitting light;
whereby the removable timer simplifies use.

11. A flexible device for light therapy, the flexible device comprising:
a flexible body;
an LED array embedded within the flexible body;
the LED array emitting therapeutic light;
a first perforated strip along a first edge of the flexible body;
a second perforated strip along a second edge of the flexible body
a first strap attachment device interfaced with the first perforated strip;
a second strap attachment device interfaced with the second perforated strip;
whereby adjusting a position of the first strap attachment device with respect to the second strap attachment device allows the flexible body to be adapted to different locations of use.

12. The flexible device for light therapy of claim 11, further comprising:
a disposable cover;
the disposable cover placed over the flexible body;
the disposable cover including one or more perforated cover edges that line up with the first perforated strip and the second perforated strip of the flexible body;
the disposable cover held in position with respect to the flexible body by the first strap attachment device and the second strap attachment device;
whereby when the first strap attachment device and the second strap attachment device are removed, the disposable cover is released from the flexible body for disposal.

13. The flexible device for light therapy of claim 11, wherein:
the first perforated strip is parallel to the second perforated strip;
the strap crossing from the first perforated strip to the second perforated strip to affix the device in position.

14. The flexible device for light therapy of claim 11, further comprising:
a disposable cover;
the disposable cover placed over the flexible body;
the disposable cover including one or more perforated cover edges that line up with the first perforated strip and the second perforated strip of the flexible body;
the disposable cover held in position with respect to the flexible body by the first strap attachment device and the second strap attachment device;
whereby when the first strap attachment device and the second strap attachment device are removed, the disposable cover is released from the flexible body for disposal.

15. The flexible device for light therapy of claim 11, further comprising:
a removable timer;
the removable timer electrically connected to the LED array;
the removable timer controlling whether the LED array is emitting light;
whereby the removable timer simplifies use.
